(19) United States
(12) Patent Application Publication
(10) Pub. No.: US 2024/0398239 A1
(43) Pub. Date: Dec. 5, 2024

(54) SYSTEM FOR MEASURING TEMPERATURE REMOTELY

(71) Applicant: VALEO SYSTEMES THERMIQUES, Le Mesnil-Saint-Denis (FR)

(72) Inventors: Georges De Pelsemaeker, Le Mesnil-Saint-Denis (FR); Mohamed-Amine Boubaker, Le Mesnil-Saint-Denis (FR); Marion Moutinho, Le Mesnil-Saint-Denis (FR)

(73) Assignee: VALEO SYSTEMES THERMIQUES, Le Mesnil-Saint-Denis (FR)

(21) Appl. No.: 18/695,561

(22) PCT Filed: Sep. 22, 2022
(86) PCT No.: PCT/EP2022/076305

§ 371 Date: Mar. 26, 2024

(30) Foreign Application Priority Data
Oct. 4, 2021 (FR) FR2110453

(51) Int. Cl. A61B 5/01; G01J 5/00; G01J 5/08; G01J 5/12

(52) U.S. Cl. CPC A61B 5/01; G01J 5/0025; G01J 5/0859; G01J 5/12; G01J 2005/0077

(57) ABSTRACT

A system for measuring temperature remotely is disclosed. The system is configured to measure a temperature on at least a zone of the face of the individual. The system includes a first temperature sensor operating in the infrared and configured to measure at least one skin temperature of the individual in at least one zone of the face of the individual, a second temperature sensor configured to measure an ambient temperature, and a processing unit. The first sensor is an infrared camera. The skin temperature measured by the first sensor is termed (Tir). The processing unit is configured to determine an intermediate skin temperature as a function at least of Tir, of a distance value indicative of the distance between the first sensor and the individual, and the ambient temperature measured by the second sensor.

The present invention relates to a system for measuring temperature remotely.

At the present time, potential COVID-19 patients are detected by taking their temperature, performing supplementary clinical examinations, then carrying out a COVID-19 test that still possesses a relatively low confidence level.

There is a great need to be able to rapidly detect, in particular by virtue of mobile means, people potentially ill with a contagious disease, for example the disease linked to COVID-19.

Temperature readings that are as reliable as possible are crucial to detecting illness in this way.

The subject matter of the invention is therefore a system for measuring temperature remotely, designed to measure a temperature on an individual, notably on at least a zone of the face of the individual, this system comprising:
a first temperature sensor operating in the infrared and designed to measure at least one skin temperature of the individual, notably in at least one zone of the face of the individual, this first sensor notably being an infrared camera, the skin temperature measured by this first sensor being termed (Tir),
a second temperature sensor designed to measure an ambient temperature,
a processing unit designed to:
   determine an intermediate skin temperature (Tbb) as a function at least of the temperature (Tir) measured by the first temperature sensor, of a distance value indicative of the distance between the first sensor and the individual, and the ambient temperature measured by the second sensor, and
   determine a corrected skin temperature value (Ts) as a function at least of the intermediate skin temperature (Tbb) and of a correction linked to the emissivity of the individual's skin.

The invention notably makes it possible to obtain a skin temperature that is as accurate as possible, with little or no interference from the distance and ambient temperature parameters. Other parameters in addition to distance and ambient temperature may be taken into consideration, as will be seen later on.

The intermediate skin temperature value (Tbb) is not calculated with skin emissivity taken into consideration. The emissivity is taken into consideration in determining the final corrected temperature (Ts), as will be seen later on.

According to one of the aspects of the invention, the system comprises a distance-measuring apparatus to supply the distance value.

As an alternative, this distance value may be a predetermined value. For example, this distance value is fixed in advance, according to the location of the chair in which the individual subject to the temperature measurement is seated, this value being fixed for example at 1 meter.

According to one of the aspects of the invention, the processing unit is designed to determine the corrected skin temperature value (Ts) from a single intermediate skin temperature value (Tbb).

According to one of the aspects of the invention, this system comprises a distance-measuring apparatus designed to measure the distance between the face of the individual and the first temperature sensor.

According to one of the aspects of the invention, this distance measurement is based for example on one zone of the face of the individual, for example on the zone of the face in which the first temperature sensor measures the skin temperature (Tir).

According to one of the aspects of the invention, this apparatus is selected from a stereoscopic measurement apparatus, a radar, an apparatus of the “time-of-flight (TOF) sensor” type, and an inertial balance which notably allows the body of the individual to be positioned on a virtual checkerboard reference grid.

According to one of the aspects of the invention, the distance measurement may also be taken by image processing by measuring the distance separating the eyes in the image and then, from this estimated separation, determining the distance between the face on which the temperature is measured and the first temperature sensor.

According to one of the aspects of the invention, the processing unit is designed to determine the corrected skin temperature value (Ts) from a plurality of intermediate skin temperature values (Tbb).

According to one of the aspects of the invention, the processing unit is designed to use the same distance value for determining all the intermediate skin temperature values (Tbb).

This, namely the use of the same distance value, may prove sufficient when the distance between the individual and the first sensor is sufficiently great, for example at least 1 meter.

As an alternative, the processing unit is designed to use several distance values for determining the intermediate skin temperature values (Tbb).

In particular, each calculation of an intermediate skin temperature value (Tbb) uses an associated distance value.

Each distance value corresponds to a point on the face of the individual. This point is a point at which the temperature measurement by the first sensor is taken.

According to one of the aspects of the invention, the first temperature sensor is designed to measure the temperatures at a plurality of points on the face of the individual.

This plurality of points notably forms a mask of measurement points laid over the face of the individual.

Measuring distance at each temperature measurement point provides better accuracy of the intermediate skin temperature values (Tbb).

This is particularly advantageous when the first temperature sensor is positioned relatively close to the face of the individual, for example at a distance of a few tens of centimeters away. Over short distances such as this, the accuracy of the value of the distance between the temperature measurement point and the first sensor has a not-insignificant influence on the accuracy of the intermediate skin temperature value (Tbb).

For example, the intermediate temperature (Tbb) at the end of the nose needs to be corrected using a distance value measured to the end of the nose. Specifically, when the first sensor is relatively close to the face, a distance measured to the end of the nose and the distance measured to another point on the face, for example the cheek, may, proportionately, differ greatly.

In the case where several distance measurements on the face are taken, the system may comprise a measurement apparatus for determining the distance at a plurality of points on the face of the individual, this apparatus being selected for example from a stereoscopic measurement apparatus or an apparatus of the “time-of-flight (TOF) sensor” type.

This camera operates on the TOF principle (TOF standing for Time of Flight) which allows a scene to be measured in 3 dimensions (3D) in real time. This type of camera is known.

By contrast, when the first sensor is positioned sufficiently far away from the individual, for example more than 70 cm away, it is possible to use the same distance value for correcting the values taken by the first sensor, so as to determine the various intermediate temperature values (Tbb). The variations in distance across the various measurement points on the face remain relatively small and do not significantly adversely affect the accuracy of the corrected temperature values.

According to one of the aspects of the invention, it comprises a camera, notably a Red Green Blue camera, or RGB camera, designed to define, on the face of the individual, the notable temperature measurement point or notable temperature measurement points, for the first sensor. This camera notably operates in the visible domain.

The first and/or the second temperature sensor comprises a component for measuring the temperature inside a housing of the sensor and/or of a sensitive part of this sensor.

For example, if the temperature measured by this sensor is outside a predetermined temperature range for which the sensor is calibrated, the system will consider the first and/or second temperature sensor as being unreliable and will disregard the data coming from that sensor.

According to one of the aspects of the invention, the correction linked to the emissivity of the individual's skin is chosen as being dependent on the intermediate skin temperature (Tbb).

According to one of the aspects of the invention, the corrected skin temperature value is given by a curve having, as abscissa, the intermediate skin temperature and, as ordinate, the skin temperature corrected to account for the emissivity of the skin.

The dependence between the emissivity of the skin and the skin temperature is contained in this curve.

This curve is obtained for example by taking actual measurements.

In one embodiment of the invention, the system does not need explicit access to a skin emissivity value. The system relies on the above-mentioned curve which contains the influence that the emissivity has on the temperature measurement in the region concerned, notably the face. The correction linked to the emissivity is contained in this curve.

According to one of the aspects of the invention, the correction linked to the emissivity of the individual's skin is chosen as being dependent also on the individual's perspiration and use of cosmetics.

According to one of the aspects of the invention, the intermediate skin temperature (Tbb) is equal to a polynomial function, for example of order 2, of the temperature (Tir) measured by the first temperature sensor, of a distance value indicative of the distance between the first sensor and the individual, and the ambient temperature measured by the second sensor.

According to one of the aspects of the invention, it comprises a light-intensity sensor, notably a sensor of thermopile type, comprising thermocouples and designed to convert thermal energy into electrical energy.

According to one of the aspects of the invention, the processing unit is connected to this light-intensity sensor, notably of thermopile type, and is designed to use light-intensity data supplied by this sensor to correct the temperature (Tbb).

The correction with respect to ambient brightness may integrate this light-intensity data divided by a radiant thermal power value associated with the basal metabolism of the individual.

According to one of the aspects of the invention, the first temperature sensor is a camera operating in the infrared, preferably at a wavelength of between 7 and 14 micrometers.

According to one of the aspects of the invention, this camera is of the digital type.

According to one of the aspects of the invention, the entire system is of the digital type, with no analog component.

According to one of the aspects of the invention, the second temperature sensor designed to measure an ambient temperature is a sensor comprising a platinum-based sensitive element, notably of PT100 type.

A further subject of the invention is a method for measuring temperature remotely, to measure a temperature on an individual, notably on at least a zone of the face of the individual, this method comprising the following steps:
supplying a first temperature sensor operating in the infrared and designed to measure at least one skin temperature of the individual, notably in at least one zone of the face of the individual, this first sensor notably being an infrared camera, the skin temperature measured by this first sensor being termed (Tir),
supplying a second temperature sensor designed to measure an ambient temperature,
determining an intermediate skin temperature (Tbb) as a function at least of the temperature (Tir) measured by the first temperature sensor, of a distance value indicative of the distance between the first sensor and the individual, and the ambient temperature measured by the second sensor, and
determining a corrected skin temperature value (Ts) as a function at least of the intermediate skin temperature (Tbb) and of a correction linked to the emissivity of the individual's skin.

According to one of the aspects of the invention, the notable point or points for the measurement of temperature are defined geometrically by means of an image zone called a building box, which surrounds it, for example by means of the geometrical average of the sides of the zone of the image. This image zone is a surface delimited by a series of points which is constructed by an object identification algorithm.

The system may be packaged inside a portable case and set up in a building, for example an airport, hospital, or at any other location.

The invention may serve to provide assistance with delivery of diagnostic information, notably with a view to detecting a disease in an individual, in particular a contagious disease such as COVID-19.

The invention and the various applications thereof will be better understood from reading the following description and from studying the accompanying figures, in which:

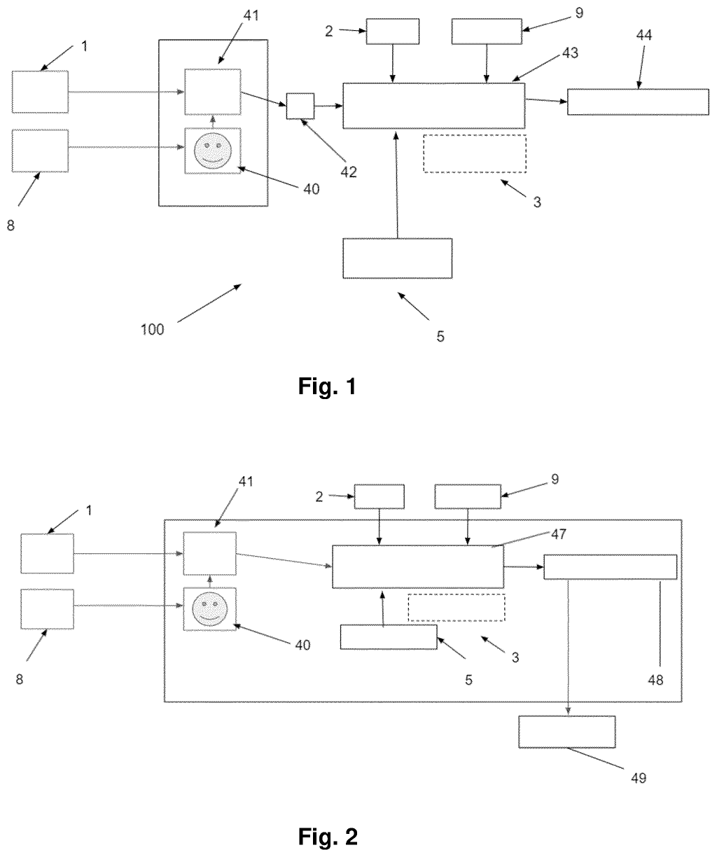

FIG. 1 schematically illustrates a system according to one non-limiting embodiment of the invention.

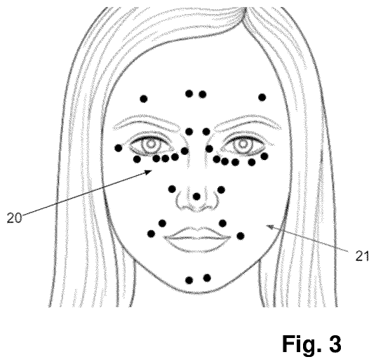

FIG. 2 schematically illustrates a system according to another non-limiting embodiment of the invention.

FIG. 3 illustrates the notable measurement points on the face of an individual, when using the system according to the invention.

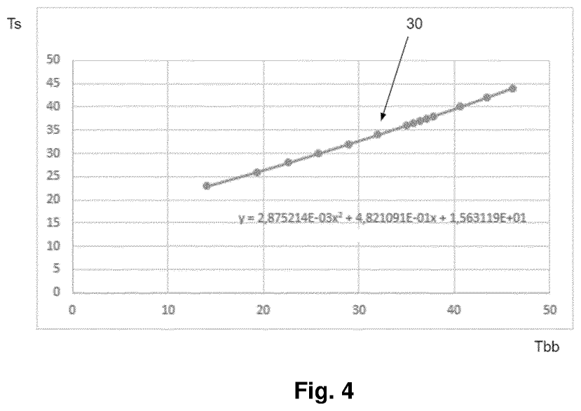

FIG. 4 shows a curve illustrating the influence that the emissivity has on the temperature measurements.

FIG. 1 depicts a system 100 for measuring temperature remotely, the system being designed to measure the temperature of an individual, in this instance on the face of the individual.

This system 100 comprises a first temperature sensor 1 operating in the infrared and designed to measure the skin temperature of the individual, at one or more zones or points on the face of the individual.

The first temperature sensor 1 is a camera operating in the infrared for example between 7 and 14 micrometers.

The skin temperature measured by this first sensor 1 is termed Tir.

This system 100 further comprises a second temperature sensor 2 designed to measure an ambient temperature Tamb.

The second temperature sensor 2 designed to measure an ambient temperature is a sensor comprising a platinum-based sensitive element, notably of PT100 type.

This system 100 comprises a processing unit 3 designed to:
determine an intermediate skin temperature Tbb as a function at least of the temperature Tir measured by the first temperature sensor 1, of a distance value Dist indicative of the distance between the first sensor 1 and the individual, and the ambient temperature Tamb measured by the second sensor 2, and
determine a corrected skin temperature value Ts as a function at least of the intermediate skin temperature Tbb and of a correction linked to the emissivity of the individual's skin.

The system 100 comprises a distance measurement apparatus 5 for supplying the distance value Dist.

As an alternative, this distance value Dist may be a predetermined value. For example, this distance value is fixed in advance, according to the location of the chair in which the individual subject to the temperature measurement is seated, this value being fixed for example at 1 meter.

In the embodiment of FIG. 1, the processing unit 3 is designed to determine the corrected skin temperature value Ts from a single intermediate skin temperature value Tbb.

The distance-measuring apparatus 5 is designed to measure the distance between the face of the individual and the first temperature sensor 1.

This distance measurement is based for example on one zone of the face of the individual, for example on the zone of the face in which the first temperature sensor 1 measures the skin temperature Tir.

This distance-measuring apparatus 5 is selected from a stereoscopic measurement apparatus, a radar, an apparatus of the “time-of-flight (TOF) sensor” type, and an inertial balance.

The distance measurement may also be taken by image processing by measuring the distance separating the eyes in the image and then, from this estimated separation, determining the distance between the face on which the temperature is measured and the first temperature sensor.

In the embodiment of FIG. 2, the processing unit 100 is designed to determine the corrected skin temperature value Ts from a plurality of intermediate skin temperature values Tbb.

In a first embodiment, the processing unit 3 is designed to use the same distance value Dist for determining all the intermediate skin temperature values Tbb.

This, namely the use of the same distance value, may prove sufficient when the distance between the individual and the first sensor 1 is sufficiently great, for example at least 1 meter.

When the first sensor is positioned sufficiently far away from the individual, it is possible to use the same distance value for correcting the values taken by the first sensor, so as to determine the various intermediate temperature values (Tbb). The variations in distance across the various measurement points on the face remain relatively small and do not significantly adversely affect the accuracy of the corrected temperature values.

In another embodiment, the processing unit 3 is designed to use a plurality of distance values Dist for determining the intermediate skin temperature values Tbb.

In particular, each calculation of an intermediate skin temperature value Tbb uses an associated distance value Dist.

Each distance value Dist corresponds to a point on the face of the individual. This point is a point at which the temperature measurement by the first sensor 1 is taken.

The first temperature sensor 1 is thus designed to measure the temperatures at a plurality of points on the face of the individual. This plurality of points notably forms a mask 20 of measurement points overlaid over the face 21 of the individual, as can be seen in FIG. 3.

Measuring distance at each temperature measurement point provides better accuracy of the intermediate skin temperature values Tbb. This is particularly advantageous when the first temperature sensor 1 is positioned relatively close to the face 21 of the individual, for example at a distance of a few tens of centimeters away. Over short distances such as this, the accuracy of the value of the distance between the temperature measurement point and the first sensor 1 has a not-insignificant influence on the accuracy of the intermediate skin temperature value Tbb. For example, the intermediate temperature Tbb at the end of the nose needs to be corrected using a distance value measured to the end of the nose. Specifically, when the first sensor is relatively close to the face, a distance measured to the end of the nose and the distance measured to another point on the face, for example the cheek, may, proportionately, differ greatly.

The number of points on the mask 20 of points may exceed 100 or even exceed 200.

This number of points on the mask is, in the example described, equal to 366 points.

In order to measure this multitude of distance values Dist, the distance-measuring apparatus is selected from a stereoscopic measurement apparatus, or an apparatus of the “time-of-flight (TOF) sensor” type.

This camera operates on the TOF principle (TOF standing for Time of Flight) which allows a scene to be measured in 3 dimensions (3D) in real time. This type of camera is known.

The system 100 comprises a camera 8, notably a Red Green Blue camera, or RGB camera, designed to define, on the face 21 of the individual, the notable temperature measurement point or notable temperature measurement points, for the first sensor 1. This is step 40 in FIGS. 1 and 2.

The notable points for the measurement of temperature are defined geometrically by means of an image zone called a building box, which surrounds it, for example by means of the geometrical average of the sides of the zone of the image. This image zone is a surface delimited by a series of points which is constructed by an object identification algorithm.

This camera 8 operates in the visible domain.

After step 40, the infrared camera 1 takes temperature measurements at these points of the mask 20 (step 41) in order to obtain the various measured temperature values Tir.

The first and/or the second temperature sensor may comprise a component for measuring the temperature inside a housing of the sensor and/or of a sensitive part of this sensor.

For example, if the temperature measured by this sensor is outside a predetermined temperature range for which the sensor is calibrated, the system will consider the first and/or second temperature sensor as being unreliable and will disregard the data coming from that sensor.

The correction linked to the emissivity of the individual's skin is chosen as being dependent on the intermediate skin temperature Tbb.

The corrected skin temperature value Ts is given by a curve 30 (see FIG. 4) having, as abscissa, the intermediate skin temperature Tbb and, as ordinate, the corrected skin temperature Ts, corrected to account for the emissivity of the skin.

The dependence between the emissivity of the skin and the skin temperature is contained in this curve 30.

In the example described, it is of order-2 polynomial form.

This curve is obtained for example by taking actual measurements.

The system does not need explicit access to a skin emissivity value. The system relies on the above-mentioned curve which contains the influence that the emissivity has on the temperature measurement in the region concerned, notably the face. The correction linked to the emissivity is contained in this curve 30.

The correction linked to the emissivity of the individual's skin may, if so desired, be chosen as being dependent also on the individual's perspiration and use of cosmetics.

The intermediate skin temperature Tbb is equal to a polynomial function, in this instance of order 2, of the temperature Tir measured by the first temperature sensor 1, of a distance value Dist indicative of the distance between the first sensor 1 and the face 21 of the individual, and the ambient temperature Tamb measured by the second sensor 2.

In the example described, the intermediate skin temperature is given by the following equation:

Tbb=a0+a1·Tir+a2·Dist+a3·Tamb+a4·Tir2+a5·Dist2+a6·Tamb2+a7·Tair·Dist+a8·Tir·Tamb+a9·Dist·Tamb

where a0, a1 . . . a9 are predetermined coefficients of the polynomial.

The system 100 comprises a light-intensity sensor 9, in this instance a sensor of thermopile type, comprising thermocouples and designed to convert thermal energy into electrical energy.

The processing unit 3 is connected to this light-intensity sensor 9, notably of thermopile type, and is designed to use light-intensity data supplied by this sensor to correct Tbb.

The correction with respect to ambient brightness may integrate this radiant light-intensity data divided by a radiant thermal power value associated with the basal metabolism of the individual.

For example, this correction with respect to ambient brightness is a corrective term proportional to the ratio between the radiant light intensity and a radiant thermal power value associated with the basal metabolism of the individual.

This proportionality coefficient is for example comprised between 0.1 and 10.

This value for the radiant thermal power associated with the basal metabolism is for example taken as being equal to 14 W·m⁻² (watts per unit area).

In addition to considering the proportionality described hereinabove, it is possible, if so desired, to force this correction for ambient brightness to be equal to zero when the radiant ambient brightness is less than 10% of the radiant thermal power associated with the basal metabolism.

The entire system 100 is of digital type, with no analog component.

In the example of FIG. 1, just one measured temperature value Tir is used to obtain the correction. The value Tir taken into consideration is the highest of the temperatures Tir on the mask of points. This step of selecting the highest value of Tir is performed in step 42 of FIG. 1.

The temperature point adopted is, for example, a point near the eye.

Step 43 seeks to determine an intermediate skin temperature Tbb as a function of the measured temperature Tir adopted, of a distance value Dist indicative of the distance between the first sensor and the individual, and the ambient temperature Tamb measured by the second sensor.

Step 43 uses the aforementioned polynomial function.

In step 44, a corrected skin temperature value Ts is determined as a function of the intermediate skin temperature Tbb and of a correction linked to the emissivity of the individual's skin, as explained above.

In the example of FIG. 2, all the temperature values Tir measured in step 41 are used to determine that same number of intermediate temperatures Tbb. It is at step 47 of FIG. 2 that 366 intermediate temperature values Tbb are obtained using the aforementioned polynomial equation.

In step 48, all these values Tbb are corrected using the emissivity correction.

In step 49, the highest of the corrected values Ts is adopted.

1. A system for measuring temperature remotely,
wherein the system is configured to measure a temperature on an individual, on at least a zone of the face of an individual, the system comprising:
a first temperature sensor operating in the infrared and configured to measure at least one skin temperature of the individual, in at least one zone of the face of the individual,
wherein the first sensor is an infrared camera,
wherein the skin temperature measured by the first sensor is termed (Tir),
a second temperature sensor configured to measure an ambient temperature, and
a processing unit configured to:
determine an intermediate skin temperature (Tbb) as a function at least of the temperature (Tir) measured by the first temperature sensor, of a distance value indicative of the distance between the first sensor and the individual, and the ambient temperature measured by the second sensor, and
determine a corrected skin temperature value (Ts) as a function at least of the intermediate skin temperature (Tbb) and of a correction linked to the emissivity of the individual's skin.

2. The system as claimed in claim 1,
wherein the system further comprises:
a distance-measuring apparatus to supply the distance value,
a stereoscopic measurement apparatus,
a radar,
an apparatus of the “time-of-flight (TOF) sensor” type, and
an inertial balance.

3. The system as claimed in claim 1,
wherein the processing unit is configured to determine the corrected skin temperature value from a plurality of intermediate skin temperature values.

4. The system as claimed in claim 3,
wherein the processing unit is configured to use the same distance value for determining all the intermediate skin temperature values.

5. The system as claimed in claim 4,
wherein the processing unit is configured to use several distance values for determining the intermediate skin temperature values.

6. The system as claimed in claim 1,
wherein the first temperature sensor is configured to measure the temperatures at a plurality of points on the face of the individual,
wherein the plurality of points form a mask of measurement points laid over the face of the individual.

7. The system as claimed in claim 1,
wherein the correction linked to the emissivity of the individual's skin is chosen as being dependent on the intermediate skin temperature.

8. The system as claimed in claim 7,
wherein the corrected skin temperature value is given by a curve having, as abscissa, the intermediate skin temperature and, as ordinate, the skin temperature corrected to account for the emissivity of the skin.

9. The system as claimed in claim 1,
wherein the system further comprises a light-intensity sensor of thermopile type,
the sensor comprising thermocouples and configured to convert thermal energy into electrical energy.

10. The system as claimed in claim 9,
wherein the processing unit is connected to light-intensity sensor of thermopile type, and
wherein the processing unit is configured to use light-intensity data supplied by the light-intensity sensor to correct the temperature.

11. A method for measuring temperature remotely, on an individual, on at least a zone of the face of the individual, the method comprising:
supplying a first temperature sensor operating in the infrared and configured to measure at least one skin temperature of the individual, in at least one zone of the face of the individual,
wherein the first sensor is an infrared camera,
wherein the skin temperature measured by the first sensor is termed,
supplying a second temperature sensor configured to measure an ambient temperature,
determining an intermediate skin temperature as a function at least of the temperature measured by the first temperature sensor, of a distance value indicative of the distance between the first sensor and the individual, and the ambient temperature measured by the second sensor, and
determining a corrected skin temperature value as a function at least of the intermediate skin temperature and of a correction linked to the emissivity of the individual's skin.
